(19) United States
(12) Patent Application Publication
(10) Pub. No.: US 2016/0068610 A1
(43) Pub. Date: Mar. 10, 2016

(54) GLUTAMATE DEHYDROGENASE IN IDH1- AND IDH2-MUTATED CANCERS

(71) Applicant: Duke University, Durham, NC (US)

(72) Inventors: Hai Yan, Chapel Hill, NC (US); Matthew Waitkus, Durham, NC (US)

(21) Appl. No.: 14/843,645

(22) Filed: Sep. 2, 2015

Related U.S. Application Data
(60) Provisional application No. 62/048,486, filed on Sep. 10, 2014.

(51) Int. Cl. C07K 16/40; A61K 31/4706; C12N 15/113; A61K 31/5383; C12Q 1/68; G01N 33/574

(52) U.S. Cl. CPC C07K 16/40; C12Q 1/6886; G01N 33/57426; G01N 33/57407; C12N 15/1135; A61K 31/5383; A61K 31/4706; C12Q 2600/158; C12N 2310/14; C07K 2317/76

(57) ABSTRACT

Expression of GLUD1, GLUD2, or both is tested in gliomas, leukemias, or suspected gliomas and leukemias. Up-regulation of expression is found in IDH1/IDH2 mutant cancers. Inhibition of GLUD1, GLUD2, or both can be used therapeutically to inhibit cancer growth. Assays for GLUD1, GLUD2, or both GLUD1 expression can measure RNA or protein or enzyme activity, for example.

This invention was made with government support under 1R01-CA 1403160 awarded by National Cancer Institute. The government has certain rights in the invention.

### TECHNICAL FIELD OF THE INVENTION

This invention is related to the area of cancer management. In particular, it relates to cancers such as gliomas and leukemias.

### BACKGROUND OF THE INVENTION

Intermediate and high-grade gliomas are malignant and lethal brain tumors for which there are relatively few efficacious therapies. Somatic mutations in the genes isocitrate dehydrogenase 1 and 2 (IDH1 and IDH2) occur in the vast majority (˜80%) of progressive gliomas and secondary glioblastomas. Wild-type IDH1/2 catalyze the conversion of isocitrate to α-ketoglutarate (aKG), but glioma-associated gene mutations that substitute Arg132 of IDH1 or Arg172 of IDH2 for a different amino acid confer a neomorphic enzyme activity in IDH1/2 proteins that catalyzes the conversion of α-ketoglutarate to D-2-hydroxyglutarate (D2HG).

D2HG is thought to be an oncometabolite that promotes gliomagenesis and leukemogenesis by inhibiting αKG-dependent dioxygenases and altering DNA methylation and epigenetics to create a cellular state permissive to malignant transformation. This view is consistent with published data from our lab and others demonstrating that IDH1/2 mutations are early, initiating events in oncogenesis. IDH1/2 mutations are thought to precede other genetic lesions and are universally expressed in malignant cells within a tumor. Therefore, IDH1/2 mutations are stable, clonal genetic lesions that are susceptible to therapeutic targeting. Consequently, inhibitors of the neomorphic activity of mutant IDH1/2 enzymes have been developed with the goal of inhibiting D2HG production and prolonging survival of patients with IDH1/2-mutant expressing tumors.

There is a continuing need in the art to develop better tools for diagnosing, prognosing, and treating cancers that have IDH1/2 mutations.

### SUMMARY OF THE INVENTION

According to one aspect of the invention a method is provided for inhibiting growth of a cancer containing a mutation in IDH1 or IDH2. An inhibitor of GLUD1, GLUD2, or both GLUD1 and GLUD2 is administered to a patient having such a cancer.

According to another aspect of the invention, a method is provided for testing patients. The test may provide information on the suitability of the patient for therapeutic agents targeting GLUD1, GLUD2, or both. The method may provide diagnostic or stratification information, identifying the patient as having an IDH1 or IDH2 mutation or suggesting a course of therapy appropriate for cancers with such expression profiles. A suspected or known glioma or suspected leukemia sample from a patient is tested for expression of GLUD1, GLUD2, or both GLUD1 and GLUD2. Normal, control cells are also tested for expression of GLUD1,GLUD2, or both GLUD1 and GLUD2. Expression in the sample from the patient is compared to the expression in the control cells. An increased expression relative to controls can be determined. Such an increase suggests that the sample contains an IDH1 or IDH2 mutation.

These and other embodiments which will be apparent to those of skill in the art upon reading the specification provide the art with new tools for managing cancers such as acute myelogenous leukemia and glioma.

### BRIEF DESCRIPTION OF THE DRAWINGS

FIGS. 1A-1D: NSCs were grown as an adherent monolayer in mouse NSC proliferation media. NSCs were fixed, permeabilized, and incubated with an anti-Nestin (green) antibody (Stem Cell Technologies). Nestin immunoreactivity was detected by an AlexaFluor-488 conjugated secondary antibody followed by fluorescence microscopy. Nuclei were counterstained with DAPI (blue). FIG. 1A: PDGFB; FIG. 1B: PDGFB-IDH1^{R132H}; FIG. 1C: PDGFB-TP53^{-/-}; FIG. 1D: PDGFB-IDH1^{R132H} TP53^{-/-}.

FIGS. 2A-2B: Histopathological analysis of murine tumors. 2.5×10⁵ NSCs were injected into the right caudate nucleus of adult NSG mice using a stereotactic device. Mice were monitored daily after injection for signs of neurological symptoms or lethargy. Symptomatic animals were euthanized according to IACUC approved protocols and 5 μm sections were stained with H&E. FIG. 2A: PGDF-TP53^{-/-}; FIG. 2B: PGDF-IDH1R132H, TP53^{-/-}.

FIG. 3: Symptom free survival of NSG mice after orthotopic transplantation of PDGFB-expressing NSCs harboring the genetic alterations indicated in the legend. P-value of a log-rank test comparing survival trends for TP53^{-/-} vs. IDH1^{R132H}-TP53^{-/-} conditions is <0.05.

FIGS. 4A-4B: Data from The Cancer Genome Atlas Low-grade Glioma dataset were analyzed for expression of GLUD1 (FIG. 4A) and GLUD2 (FIG. 4B) mRNA. Samples included in the analysis were 262 tumors for which there was mutation and gene expression data as of Sep. 7, 2014. Data are expressed as the log2 (RPKM fold change) relative to the median value of all tumors.

FIG. 5: PDGFB-IDH1^{R32H}-TP53^{-/-} NSCs were infected with empty vector retrovirus, GLUD1-expressing retrovirus, or GLUD2-expressing retrovirus. 2250 NSCs were seeded into a collagen gel in the presence of high glucose (4500 mg/L) or low-glucose (450 mg/L). Colonies were grown over a period of 14 days. Plates were imaged and then quantified using ImageJ software. Top labels indicate the transgene expressed.

FIG. 6: Western blot analysis showing expression of GLUD1/2 following lentiviral-mediated infection with control non-targeting shRNA or GLUD1/2-targeting shRNA.

FIG. 7: 1×10⁵ 08-0537 cells were injected into the right caudate nucleus of adult Nude mice using a stereotactic device. Mice were monitored daily after injection for signs of neurological symptoms or lethargy. Symptomatic animals were euthanized according to IACUC approved protocols.

### DETAILED DESCRIPTION OF THE INVENTION

The inventors have developed methods for testing and treating cancers, particularly certain brain cancers and leukemias that have certain mutations in IDH1/2. The inventors have found that expression of IDH1 mutant protein significantly slows the growth of gliomas in a mouse model.

A second consequence of mutant IDH1/2 proteins is widespread reprogramming of tumor cell metabolism. In particular, IDH1/2 mutations alter the abundance of amino acids and promote reductive glutamine metabolism under hypoxic conditions. As an alternative therapeutic strategy, we hypothesized that targeting altered metabolic pathways in mutant IDH tumors may effectively inhibit their growth.

Two potential targets for metabolic disruption of IDH1/2 mutant tumors are glutamate dehydrogenase 1 and 2 (GLUD1, GLUD2). These enzymes are highly expressed in IDH1 and IDH2 mutant gliomas relative to IDH wild-type tumors, and GLUD 1/2 normally function to replenish cellular αKG levels by converting 1-glutamate to αKG. Importantly, expression of GLUD2 increases the growth of IDH1 mutant-expressing neural stem cells. Further, targeting GLUD1 and GLUD2 by RNA interference significantly slowed the growth of an IDH1 R132H mutant xenograft tumor in a mouse model.

Targeting enzymes involved in altered metabolic pathways of IDH1/2 mutant-expressing tumors, in particular GLUD1 and GLUD2, significantly inhibits tumor growth. In established tumors, IDH1/2 mutations significantly alter cellular metabolism and likely require GLUD1/2 activity to maintain a high rate of cell growth and proliferation. The GLUD1/2 pathway is therefore a metabolic Achilles heel of IDH mutant tumors that can be targeted to inhibit tumor growth.

Cancers to which the methods described here may be applied include, without limitation, diffuse gliomas, including diffuse astrocytoma, anaplastic astrocytoma, oligodendroglioma, anaplastic oligodendroglioma, oligoastrocytoma, anaplastic oligoastrocytoma, secondary glioblastoma, and acute myeloid leukemia. Any cancer in which IDH1/2 mutations are present may be subject to these methods.

Inhibitors which can be used to inhibit growth of this type of tumors includes without limitation, antibodies which specifically bind to GLUD1, GLUD2, or both. Antibodies which can be used include, without limitation, polyclonal, monoclonal, single chain, antibody fragments such as Fab′ and F(ab′)₂, bi-specific antibodies, etc. Inhibitors can also be small molecule inhibitors, such as R162 and chloroquine. Inhibitors can be inhibitory nucleic acids, such as shRNA, RNAi, antisense nucleic acid, and antisense vectors. Any inhibitor may be used for these enzymes, whether they inhibit expression or function.

Cancers for treatment may be selected based on prior testing and identification of IDH1/2 mutations, or up-regulation of expression of GLUD1, GLUD2, or both. Typically the cancer will be one of the types which are known to commonly have IDH1/IDH2 mutations. Mutations can be tested using any known technique including but not limited to sequence determination, hybridization to a probe, allele-specific amplification, amplification followed by an allele-specific probe, single base extension reactions, antibody testing for a neoepitope, etc. Comparison to a control tissue or nucleic acid sample can indicate mutation relative to wild type or up-regulation relative to base line expression. Comparison to reference expression data may also be used.

Expression testing of GLUD1, GLUD2, or both, can be performed by any method known in the art. These may include without limitation, serial analysis of gene expression, RNA hybridization and quantitation, enzyme assay, immunoblotting, and quantitative RT-PCR. Any method for analyzing RNA or protein from the genes for GLUD1, GLUD2, or both may be used.

Diagnostic methods can be combined with therapeutic methods if desired, so that a single workflow includes both parts. Alternatively, each method can be performed separately. It may be desirable to test both for up-regulation of GLUD1, GLUD2, or both, as well as for mutation in IDH1/2. Both these tests may be combined with the therapeutic method.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

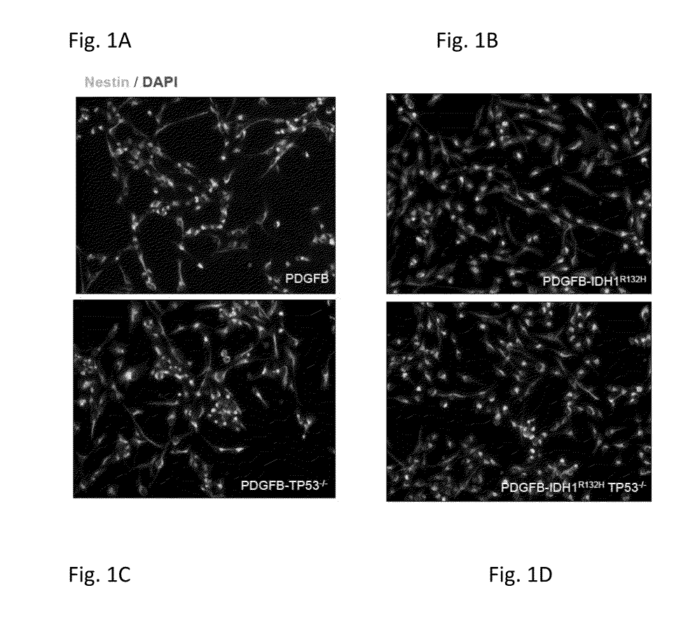

We have generated a library of murine neural stem cells (NSCs) that are genetically engineered to harbor and express cancer-related genetic alterations. We have developed NSC lines that have IDH1^{R132H} mutation, TP53 deletion (TP53^{-/-}) or both. We have also generated wild-type NSC controls. All of these lines stain positive for the NSC and glial progenitor marker nestin using immunofluorescence (FIG. 1). Furthermore, all of these lines express a human PDGFB transgene that functions as an oncogenic driver.

### EXAMPLE 2

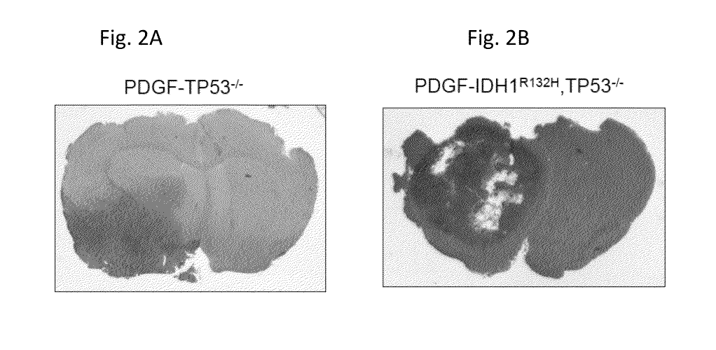

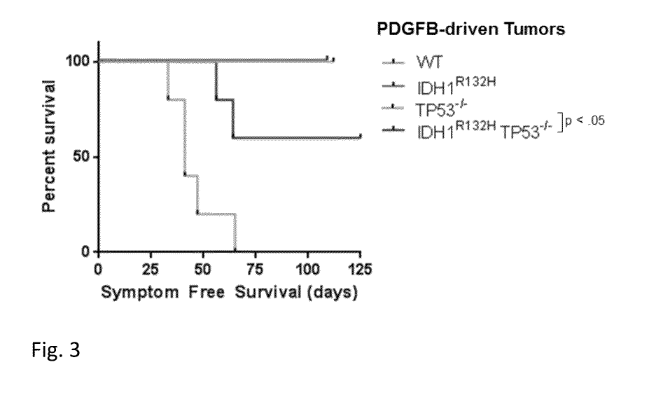

The TP53^{-/-} NSC line and IDH1^{R132H}-TP53^{-/-} NSC line are tumorigenic following orthotopic transplantation into the right caudate nucleus of NOD-SCID-γ (NSG) mice (FIG. 2). However, IDH1^{R132H} expression in TP53^{-/-} NSCs causes a delayed onset of symptoms relative to tumors bearing only TP53^{-/-}, suggesting that growth is slowed in IDH1^{R132H}-expressing tumors (FIG. 3).

### EXAMPLE 3

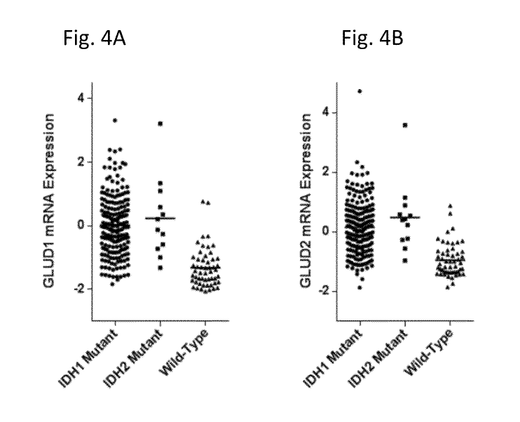

One possible mechanism for slowed growth of IDH1^{R132H}-expressing tumors is a diverted flux of αKG, a key TCA cycle metabolite and precursor of macromolecular biosynthesis, to the oncometabolite 2HG. Therefore, mechanisms that increase cellular αKG may promote growth in IDH-mutant tumors. Analysis of The Cancer Genome Atlas Low Grade Glioma RNA-sequencing gene expression data revealed that the mRNA expression of the GLUD1 and GLUD2 genes is highly elevated in IDH mutant tumors relative to IDH wild-type tumors (FIG. 4). GLUD1 and GLUD2 catalyze the conversion of the 1-glutamate, an amino acid and neurotransmitter, to αKG. Therefore, GLUD1 and GLUD2 may replenish αKG levels in IDH mutant tumors to compensate for the diverted flux of αKG to 2HG by mutant IDH enzymes.

### EXAMPLE 4

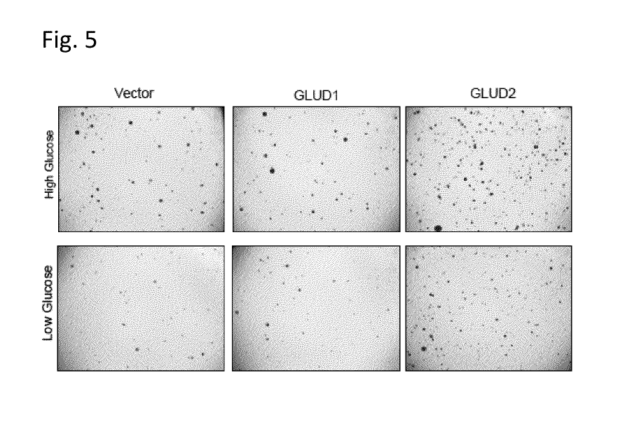

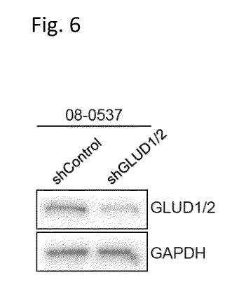

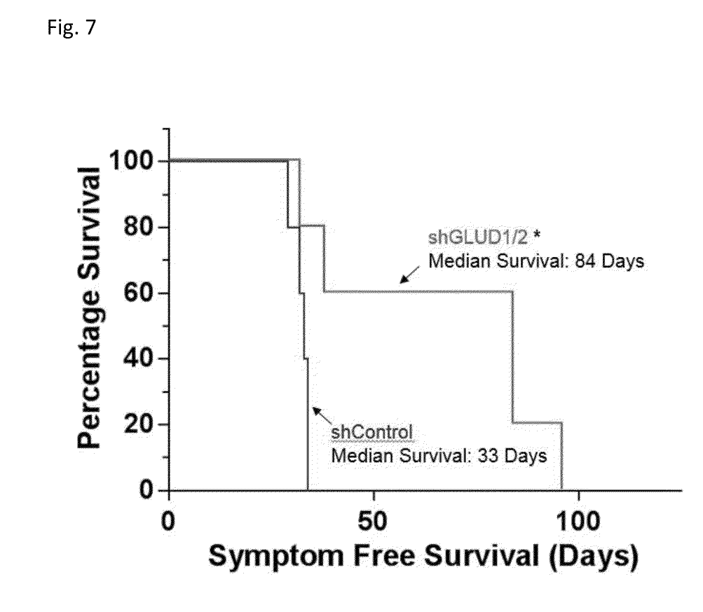

In the presence of L-glutamate, expression of GLUD2, but not GLUD1, increases IDH1^{R132H}-TP53^{-/-} NSC colony formation in collagen gel when grown in both high glucose and low glucose condition (FIG. 5). Furthermore, we used RNAi to deplete GLUD1/2 protein levels in a patient-derived glioma cell line (FIG. 6) that expresses IDH1^{R132H} mutation (08-0537 cells). NSG mice injected intracranially with 08-0537 cells expressing GLUD1/2-targeting shRNA have a longer symptom free survival compared to mice injected with cells expressing non-targeting shRNA (FIG. 7), suggesting that GLUD1/2 positively regulate the growth of this glioma cell line.

### REFERENCES

Any document cited is incorporated by reference in its entirety.
1. Mardis E R, et al. (2009). “Recurring mutations found by sequencing an acute myeloid leukemia genome”. N. Engl. J. Med. 361 (11): 1058-66.
2. Parsons D W, et al. (September 2008). “An integrated genomic analysis of human glioblastoma multiforme.”. Science 321 (5897): 1807-12.
3. Yan H, et al. (February 2009). “IDH1 and IDH2 mutations in gliomas.”. N Engl J Med 360 (8): 765-73.
4. Jin L, et al., (February 2015) “Glutamate dehydrogenase 1 signals through antioxidant glutathione peroxidase 1 to regulate redox homeostasis and tumor growth.” Cancer Cell. 27(2):257-70

We claim:

1. A method for inhibiting growth of a cancer containing a mutation in IDH1 or IDH2, comprising:
administering to a patient having said cancer an inhibitor of GLUD1,GLUD2, or both GLUD1 and GLUD2.

2. The method of claim 1 wherein the cancer is a glioma.

3. The method of claim 1 wherein the cancer is acute myelogenous leukemia (AML).

4. The method of claim 1 wherein the cancer contains an IDH1 mutation.

5. The method of claim 1 wherein the cancer contains an IDH2 mutation.

6. The method of claim 1 wherein the cancer contains an IDH1 R132H mutation.

7. The method of claim 1 wherein the cancer contains an IDH2 R172H mutation.

8. The method of claim 1 wherein the inhibitor is a small molecule inhibitor.

9. The method of claim 1 wherein the inhibitor is an antibody which specifically binds to GLUD1 and/or GLUD2 and inhibits its enzymatic activity.

10. The method of claim 1 wherein the inhibitor is an inhibitory RNA molecule which binds specifically to GLUD1 and/or GLUD2 mRNA.

11. The method of claim 1 wherein the inhibitor is an shRNA molecule which binds specifically to GLUD1 and/or GLUD2 mRNA.

12. The method of claim 1 wherein the inhibitor is R162.

13. The method of claim 1 wherein the inhibitor is chloroquine.

14. A method comprising:
testing a suspected glioma or suspected leukemia sample, or a glioma or leukemia sample, from a patient for expression of GLUD1, GLUD2, or both GLUD1 and GLUD2;
testing normal, control cells for expression of GLUD1,GLUD2, or both GLUD1 and GLUD2; and
determining an increased expression in GLUD1,GLUD2, or both GLUD1 in the suspected glioma or leukemia sample.

15. The method of claim 14 further comprising the step of administering to the patient an inhibitor of GLUD1, GLUD2, or both GLUD1 and GLUD2.

16. The method of claim 15 wherein the inhibitor is a small molecule inhibitor.

17. The method of claim 15 wherein the inhibitor is an antibody which specifically binds to GLUD1 and/or GLUD2 and inhibits its enzymatic activity.

18. The method of claim 15 wherein the inhibitor is an inhibitory RNA molecule which binds specifically to GLUD1 and/or GLUD2 mRNA.

19. The method of claim 15 wherein the inhibitor is an shRNA molecule which binds specifically to GLUD1 and/or GLUD2 mRNA.

20. The method of claim 15 wherein the inhibitor is R162.

21. The method of claim 15 wherein the inhibitor is chloroquine.

22. The method of claim 14 wherein expression is tested using an antibody which is specific for GLUD1, GLUD2, or both GLUD1 and GLUD2.

23. The method of claim 14 wherein expression is tested using an enzyme assay for activity of GLUD1, GLUD2, or both GLUD1 and GLUD2.

24. The method of claim 14 wherein expression is tested by assaying mRNA encoding GLUD1, GLUD2, or both GLUD1 and GLUD2.
